(19) United States
(12) Patent Application Publication
(10) Pub. No.: US 2025/0195209 A1
(43) Pub. Date: Jun. 19, 2025

(54) HEMOSTASIS VALVE FOR USE WITH THROMBECTOMY APPARATUSES

(71) Applicant: Boston Scientific Scimed, Inc., Maple Grove, MN (US)

(72) Inventors: Michael Dotsey, Chester Springs, PA (US); Andrew Miller, Phoenixville, PA (US); Jayson Delos Santos, Alamo, CA (US); Michael Turovskiy, San Leandro, CA (US)

(73) Assignee: Boston Scientific Scimed, Inc., Maple Grove, MN (US)

(21) Appl. No.: 18/980,685

(22) Filed: Dec. 13, 2024

Related U.S. Application Data
(60) Provisional application No. 63/610,929, filed on Dec. 15, 2023.

(51) Int. Cl. A61F 2/24

(52) U.S. Cl. CPC A61F 2/2409; A61F 2/2487

(57) ABSTRACT

A hemostasis valve includes a first hub and a second hub arranged with respective faces facing each other. An elastomeric tube extends between the first hub and the second hub and includes an inner surface defining part of a device lumen extending through the housing, and a plurality of cord segments extending from the first face to the second face, each of the plurality of cord segments circumferentially spaced from neighboring cord segments. Relative rotation between the first hub and the second hub causes each of the plurality of cord segments to cinch down onto an outer surface of the elastomeric tube and compress the elastomeric tube down into the device lumen, thereby sealing between the inner surface of the elastomeric tube and any elongate device extending through the device lumen.

### CROSS-REFERENCE TO RELATED APPLICATIONS

This application claims the benefit of priority under 35 U.S.C. § 119 of U.S. Provisional Application No. 63/610,929, filed Dec. 15, 2023, the entire disclosure of which is hereby incorporated by reference.

### TECHNICAL FIELD

The present disclosure pertains to medical devices, and methods for manufacturing and using medical devices. More particularly, the present disclosure pertains to medical devices, methods, and systems for performing thrombectomy procedures.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a hemostasis valve adapted to allow an elongate device to pass through the hemostasis valve while sealing against the elongate device. The hemostasis valve includes a housing. The housing surrounds a first hub having a first face and a second hub having a second face, the second hub arranged such that the second face faces the first face. An elastomeric tube extends between the first hub and the second hub and includes an outer surface and an inner surface defining part of a device lumen extending through the housing. A plurality of cord segments extend from the first face to the second face and each of the plurality of cord segments are circumferentially spaced from neighboring cord segments. Relative rotation between the first hub and the second hub causes each of the plurality of cord segments to cinch down onto the outer surface of the elastomeric tube and compress the elastomeric tube down into the device lumen, thereby sealing between the inner surface of the elastomeric tube and any elongate device extending through the device lumen.

Alternatively or additionally, the first hub may be stationary.

Alternatively or additionally, the first hub may include an annular recess having a bottom surface, the bottom surface defining the first face.

Alternatively or additionally, the second hub may be adapted to be rotated.

Alternatively or additionally, the second hub may include an annular protrusion that is adapted to extend into the annular recess of the first hub and an annular periphery adjacent the annular protrusion, the annular periphery defining the second face.

Alternatively or additionally, the second hub may include a hub geared surface.

Alternatively or additionally, the hemostasis valve may further include a lever including an elongate lever body extending from a first region to a second region. The first region is pivotably secured to the housing and the second region includes an actuation member extending away from the elongate lever body and extending into the housing. The actuation member includes an actuation member gear surface adapted to engage the hub geared surface such that moving the lever relative to the housing causes rotation of the second hub as the actuation member gear surface translates relative to the hub gear surface.

Alternatively or additionally, the lever may be movable between a first position in which the second hub remains stationary and a second position in which the second hub is caused to rotate.

Alternatively or additionally, the lever may be biased to the first position.

Alternatively or additionally, the hemostasis valve may exhibit minimal insertion force for inserting the elongate device into the lumen when the lever is in the first position.

Alternatively or additionally, the hemostasis valve may further include a third hub positioned such that the second hub is disposed between the first hub and the third hub, the second hub further including a cylindrical body that extends into an annular void formed within the third hub. The second hub rotates between the first hub and the third hub.

Alternatively or additionally, each of the plurality of cord segments may include a portion of one or more cords extending back and forth between the first hub and the second hub.

Another example may be found in a hemostasis valve. The hemostasis valve includes a stationary hub and a rotatable hub. The stationary hub includes an annular recess having a bottom surface and a stationary hub lumen extending through the stationary hub. The rotatable hub includes an annular protrusion that extends into the annular recess, an annular periphery adjacent the annular protrusion, and a rotatable hub lumen extending through the rotatable hub lumen. A plurality of cord segments extend between the bottom surface of the stationary hub and the annular periphery of the rotatable hub and a polymeric member extends through the stationary hub lumen and the rotatable hub lumen, the polymeric member defining a device lumen extending therethrough. Rotating the rotatable hub causes each of the plurality of cord segments to cinch down onto the polymeric member and compress the polymeric member down into the device lumen, thereby sealing between an inner surface of the polymeric member and any elongate device extending through the lumen.

Alternatively or additionally, the bottom surface of the annular recess may include a plurality of apertures adapted to accept each of the plurality of cords.

Alternatively or additionally, the annular periphery may include a plurality of apertures adapted to accept each of the plurality of cords.

Alternatively or additionally, the hemostasis valve may further include a housing that houses the stationary hub, the rotatable hub, the plurality of cords and the polymeric member.

Alternatively or additionally, the rotatable hub may further include a hub geared surface.

Alternatively or additionally, the hemostasis valve may further include a lever including an elongate lever body extending from a first end to a second end. The first end is pivotably secured to the housing and the second end includes an actuation member extending away from the elongate lever body and extending into the housing. The actuation member includes an actuation member gear surface adapted to engage the hub geared surface such that moving the lever relative to the housing causes rotation of the second hub as the actuation member gear surface translates relative to the hub gear surface.

Another example may be found in a hemostasis valve. The hemostasis valve includes a first stationary hub, a second stationary hub spaced from the first stationary hub, a rotatable hub rotatably supported by the first stationary hub and the second stationary hub, one or more cords extending back and forth between the first stationary hub and the rotatable hub, and a polymeric member extending through the first stationary hub, the second stationary hub and the rotatable hub, the polymeric member defining a device lumen extending therethrough. Rotating the rotatable hub causes each of the plurality of cords to cinch down onto the polymeric member and compress the polymeric member down into the device lumen, thereby sealing between an inner surface of the polymeric member and any elongate device extending through the lumen.

Alternatively or additionally, the hemostasis valve may further include a lever that, when actuated, causes the rotatable hub to rotate.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A shows an inverting tube (e.g., thrombectomy) apparatus that may be used to remove material from a vessel, shown in a side view with an inversion support catheter and a flexible outer tube;

FIG. 1B shows an inverting tube (e.g., thrombectomy) apparatus that may be used to remove material from a vessel, shown in a vessel, proximal to a clot;

FIG. 1C illustrates the removal of a clot from the vessel using the apparatus of FIG. 1A, by pulling the flexible tube on the outside of the inversion support catheter proximally so that it rolls over the distal end of the inversion support catheter and into the inversion support catheter, drawing the clot with it;

FIG. 2A shows an inverting tube apparatus including an expandable funnel that is attached to a distal end of an inversion support catheter;

FIG. 2B shows the inverting tube apparatus of FIG. 2A in a deployed configuration with an intermediate (e.g., delivery) catheter withdrawn proximally so that the expandable funnel at the distal end of the inversion support catheter can expand;

FIG. 3 is a perspective view of an illustrative hemostasis valve usable with the thrombectomy apparatuses of FIGS. 1A-1C and 2A-2B;

FIG. 4 is a perspective view of the illustrative hemostasis valve of FIG. 3, shown with a housing removed;

FIG. 5 is a perspective view of the illustrative hemostasis valve of FIG. 3, showing an illustrative actuation mechanism;

FIG. 6 is a perspective end view of a portion of the illustrative actuation mechanism shown in FIG. 5;

FIG. 7 is a perspective view of a portion of the illustrative actuation mechanism;

FIG. 8 is a cross-sectional view taken along the line 8-8 of FIG. 7;

FIGS. 9A and 9B provide various angle perspective views of the portion of the illustrative actuation mechanism shown in FIG. 7;

FIG. 10 is a perspective view of an illustrative hemostasis valve usable with the thrombectomy apparatuses of FIGS. 1A-1C and 2A-2B;

FIG. 11 is a perspective view of the illustrative hemostasis valve of FIG. 10, shown with part of the housing removed;

FIG. 12 is an exploded perspective view of an illustrative actuation mechanism forming part of the illustrative hemostasis valve of FIG. 10;

FIG. 13 is a schematic view of an illustrative hemostasis valve within a user's hand;

FIG. 14 is a perspective view of part of an illustrative actuation mechanism, shown prior to assembly of a hemostasis valve including the actuation mechanism;

FIG. 15A is a perspective view of part of the illustrative actuation mechanism, shown after assembly of a hemostasis valve including the actuation mechanism;

FIG. 15B is a side view of the illustrative actuation mechanism shown in FIG. 15A; and

FIG. 16 is an end view of part of an illustrative actuation mechanism.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The methods and apparatuses described herein may also relate to improvement in the operation, and in particular, the insertion and use of, inverting tube apparatuses for removing material from within a body. These apparatuses may generally include an inversion support, which may include a catheter and in some examples a funnel region at the distal end of the catheter, a flexible tube configured to move over the outside of the inversion support and invert into the inversion support, and in some examples a puller attached to a first end of the inversion support for pulling the flexible tube into the inversion support. These apparatuses may be generally referred to as apparatuses for removing a material from a vessel and may be configured as mechanical thrombectomy apparatuses.

Also described herein are systems and methods for improving the ability of the flexible tube to grab and extract material from the walls of the body lumen. Also described herein are methods for enhancing or improving the ability of the apparatus to draw in a clot by creating slack into the flexible tube before it inverts over the distal end of the inversion support catheter.

Any of these features, components and techniques may be used separately or in combination.

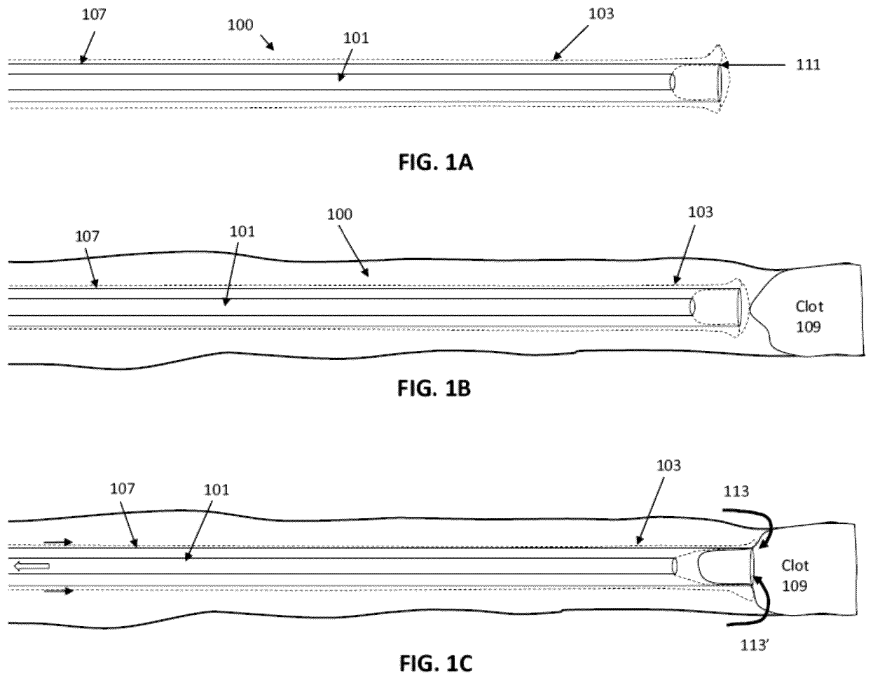

In general, an inverting tube apparatus (also referred to herein as “mechanical thrombectomy apparatus” or “inverting thrombectomy apparatus”) may be configured to remove material, such as a clot, using a length of inverting tube, as shown in FIGS. 1A-IC. The apparatuses and methods of using them described herein may be used within the vasculature, including the neuro-vasculature and the peripheral vasculature.

For example, FIG. 1A provides an example of an inverting thrombectomy apparatus 100, such as described in U.S. Pat. No. 10,028,759, and in U.S. Pat. No. 9,463,035, the entireties of which are incorporated by reference. The apparatus includes an inversion support catheter 107 and a flexible tube 103 that extends over the outer surface of the inversion catheter 107. The flexible tube 103 may be referred to as a tractor tube (or flexible tractor tube) and may be attached at one end region to a puller 101, which may be a pull wire or a pull tube (e.g., catheter), e.g., at the distal end region of the puller 101. In some examples the flexible tube 103 may be attached proximal to the distal end of the puller 101 (e.g., between 1 mm (0.04 inches) and 50 mm (1.97 inches) from the distal end, between 1 mm and 40 mm (1.57 inches), between 1 mm and 30 mm (1.18 inches), greater than 5 mm (0.30 inches), greater than 10 mm (0.39 inches), greater than 20 mm (0.79 inches), greater than 30 mm, etc. from the distal end of the puller). Pulling the puller 101 proximally inverts the flexible tube 103 over the distal end opening 111 of the inversion support catheter 107 to capture and remove a material (such as a clot) in the vessel lumen, as shown in FIGS. 1B and 1C. In operation, the amount of the material that may be captured corresponds to the length of the flexible tube 103.

In FIG. 1B, the inverting thrombectomy apparatus 100 is shown deployed near a clot 109. In the deployed configuration the puller 101 (shown here as a puller micro catheter, alternatively the puller 101 may be a wire) is held within an elongate inversion support catheter 107 so that the flexible tractor tube 103 extends from the end of the puller 101 and expands toward the inner radius of the elongate inversion support catheter 107. At the distal end opening 111 (designated in FIG. 1A) of the elongate inversion support catheter 107 the tractor tube 103 inverts over itself and extends proximally in an inverted configuration over the distal end of the elongate inversion support catheter 107. As shown in FIG. 1C, by pulling the puller 101 proximally, the tractor tube 103 inverts over the distal end opening 111 of the elongate inversion support catheter 107, inverting in a direction indicated by arrows 113 and 113′, drawing the adjacent clot 109 into the elongate inversion support catheter 107, as shown.

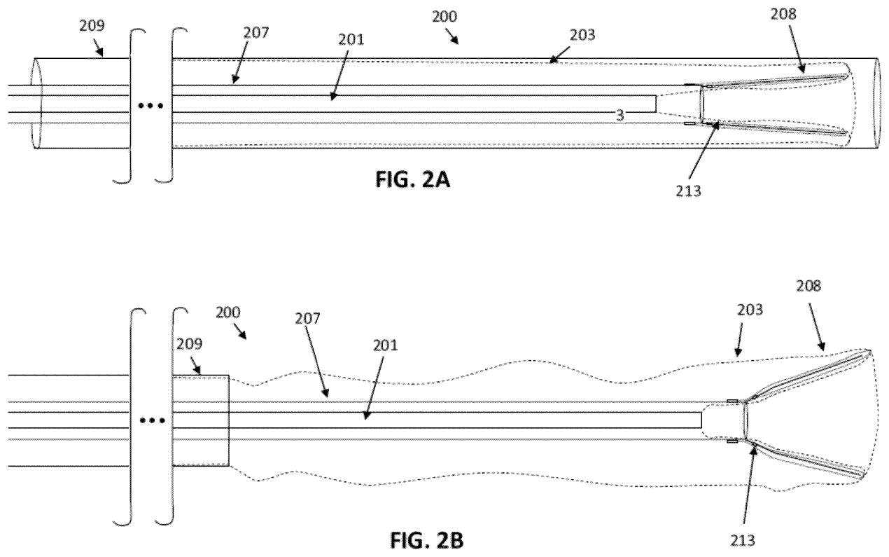

In FIG. 1A, the elongate inversion support catheter 107 is an elongate tube having a distal end that has the same size inner diameter as the proximal end of the inversion support catheter 107. In some examples, the distal end of the inversion support catheter 107 may be funnel-shaped (or configured to expand into a funnel shape, see, e.g. FIGS. 2A-2B). In FIGS. 1A-1C, the inversion support catheter 107 is shown positioned between the tractor tube (e.g., flexible tube 103) and the puller 101 so that the flexible tube 103 can be pulled proximally by pulling on the puller 101 and rolling the flexible tube 103 into the elongate inversion support catheter 107 so that the flexible tube 103 inverts. The portion of the flexible tube 103 that is inverted over the distal end of the elongate inversion support catheter 107 has an outer diameter that is greater than the outer diameter of the elongate inversion support catheter 107.

The flexible tube 103 may be biased so that it has a relaxed expanded configuration with a diameter that is greater than the outer diameter (OD) of the elongate inversion support catheter 107. In addition, the flexible tube 103 may also be configured (e.g., by heat setting, etc.) so that when the flexible tube 103 is inverted and rolled over the distal end opening into the elongate inversion support catheter 107, the outer diameter of the flexible tube 103 within the elongate inversion support catheter 107 has an outer diameter that is about y times (y fold) the inner diameter of the elongate inversion support catheter 107 (e.g., where y is greater than 0.1×, 0.5×, 0.6×, 0.7×, 0.75×, 0.8×, 0.9×, 1×, etc. the inner diameter, ID, of the elongate inversion support catheter 107. This combination of an un-inverted diameter of the flexible tube 103 of greater than the diameter of the OD of the elongate inversion support catheter 107 and an inverted diameter of the flexible tube of greater than, e.g., 0.7× the ID of the elongate inversion support catheter 107 is surprisingly helpful for preventing jamming of the apparatus, both when deploying the apparatus and when rolling the flexible tube 103 over the distal end opening 111 of the elongate inversion support catheter 107 to draw in a clot. The flexible tube 103 may be expandable and may be coupled to the puller 101 as shown. In some examples the flexible tube 103 and the puller 101 may be made of the same material, but the flexible tube may be more flexible and/or expandable, or may be connected to the elongate puller 101 (e.g., a push/pull wire or catheter). In some instances, the flexible tube 103 may be more flexible because the flexible tube 103 has a thinner material thickness relative to the elongate puller 101. In some instances, the flexible tube 103 may be more flexible than the elongate puller 101 because the flexible tube 103 may be made of a lower durometer version of a material while the elongate puller 101 is made of a higher durometer version of the same material. This is just an example. As mentioned above, the elongate puller 101 may be optional (e.g., the flexible tube 103 may itself be pulled proximally into the inversion support catheter 107).

As seen in FIG. 1C, the clot may be drawn into the elongate inversion support catheter 107 by pulling the flexible tube 103 proximally into the distal end of the elongate inversion support catheter 107, as indicated by the arrows 113, 113′ showing pulling of the inner portion of the flexible tube 103, resulting in rolling the flexible tube 103 over the distal end opening 111 of the catheter 107 and into the catheter distal end and inverting the expandable distal end region so that the flexible tube 103 is pulled into the inversion support catheter 107, shown by arrows 113 and 113′. The end of the flexible tube 103 outside of the inversion support catheter 107 may be loose relative to the outer wall of the inversion support catheter 107, meaning that there is sufficient clearance between the flexible tube 103 and the catheter 107 so that the flexible tube 103 easily moves relative to the inversion support catheter 107.

FIGS. 2A-2B illustrate an example of an inverting thrombectomy apparatus 200 that includes a funnel region at the distal end of an inversion support catheter. In this example, the inverting thrombectomy apparatus 200 includes an elongate, flexible inversion support catheter 207 that has an expandable funnel 208 at the distal end, shown in a collapsed configuration in FIG. 2A within an intermediate (e.g., delivery) catheter 209, and in an open configuration in FIG. 2B after being released from the intermediate catheter 209. The funnel 208 may be formed of a woven material and may be porous, particularly at a base region 213, where the funnel 208 extends from the inversion support catheter 207. A flexible tube 203 extends over the distal end (including the funnel 208) of the inversion support catheter 207 and inverts over the distal opening of the funnel 208. The flexible tube 203 may be, e.g., a knitted material, and may be biased to expand to an outer diameter (OD) that is larger than the OD of the funnel 208 in the open configuration. The flexible tube 203 is attached to a distal end region of a puller 201. The flexible tube (e.g., “tractor”) is attached to the distal end region of the puller 201. The funnel 208 may include two or more regions having different wall angles.

It will be appreciated that during use of the inverting thrombectomy apparatus 100 or the inverting thrombectomy apparatus 200 that it may be necessary to advance one element within another element. For example, the elongate inversion support catheter 107 may be advanced through an intermediate (e.g., delivery) catheter (not shown in FIGS. 1A-IC). There is a desire to be able to easily insert and advance the elongate inversion support catheter 107 into the intermediate catheter while still being able to seal between the elongate inversion support catheter 107 and the intermediate catheter. With respect to FIGS. 2A and 2B, there is a desire to be able to easily insert and advance the elongate inversion support catheter 207 into the intermediate catheter 209 while still being able to seal between the elongate inversion support catheter 207 and the intermediate catheter 209. In some instances, a hemostasis valve may be used with the intermediate catheter (such as the intermediate catheter 209) that is adapted to permit a low or even zero insertion force for inserting another elongate device (such as the elongate inversion support catheter 107 and 207) through the hemostasis valve and into the intermediate catheter when the hemostasis valve is in an open configuration while being able to seal well against the inserted elongate device when in a closed configuration.

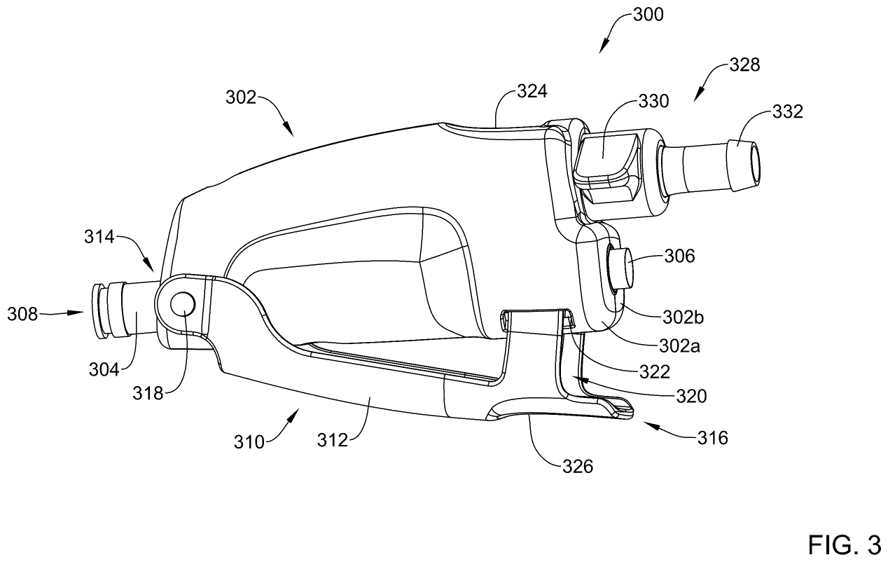

FIG. 3 is a perspective view of an illustrative hemostasis valve 300 that may be used, for example, in combination with the elongate inversion support catheter 107/207 and the intermediate catheter 209. However, the illustrative hemostasis valve 300 may be used in combination with any of a variety of different elongate medical devices such as vascular access sheathes for sealing around any of a variety of various-sized ancillary devices such as guidewires, catheters, sheathes, and the like. The elongate inversion support catheter 107 (or the elongate inversion support catheter 207) are merely an example of a device that may be inserted through the hemostasis valve 300 and thus into an intermediate catheter, including the intermediate catheter 209. In some instances, the hemostasis valve 300 may be adapted to seal under both negative pressures and positive pressures.

The hemostasis valve 300 includes a housing 302 that in some instances may include a left hand housing 302a and a right hand housing 302b. It will be appreciated that reference to left hand and right hand refer to the illustrated orientation, and is not intended to provide any limitation as to the orientations in which the hemostasis valve 300 may be used, particularly since the hemostasis valve 300 is adapted to function regardless of its orientation. A tubular member 304 extends distally from the housing 302. A polymeric member 306, which (as will be described) forms part of an actuation mechanism for selectively opening or closing the hemostasis valve 300, is fluidly coupled with the tubular member 304 such that the tubular member 304 and the polymeric member 306 together form a device lumen 308 that extends through the hemostasis valve 300 and that is adapted to accept any of a variety of devices through the device lumen 308. The polymeric member 306 may be an elastomeric tube, for example. A variety of different polyurethane materials may be used for making the polymeric member 306.

The hemostasis valve 300 includes a lever 310 having an elongate lever body 312 that extends from a first region 314 to a second region 316. In some instances, the first region 314 may be pivotably secured to the housing 302 at a pivot point 318. In some instances, the second region 316 may include an actuation member 320 extending away from the elongate lever body 312 and extending into the housing 302. In some instances, the housing 302 may include apertures 322 that are dimensioned and positioned to accommodate the actuation member 320. The actuation member 320 may include one element extending into the housing 302. In some instances, as shown, the actuation member 320 may include two parallel elements extending into the housing 302.

In some instances, the housing 302 and the lever 310 may together be adapted to be ergonomic in improving an ability for a user to grasp the housing 302 and squeeze the lever 310 towards the housing 302 in order to cause the hemostasis valve 300 to transition from an open configuration to a closed configuration. Accordingly, the housing 302 may include a recessed region 324 and/or the lever 310 may include a recessed region 326. In some instances, the housing 302 may include the recessed region 324 but the lever 310 may not include the recessed region 326. In some instances, the lever 310 may include the recessed region 326 but the housing 302 may not include the recessed region 324.

The lever 310 may be movable between a first position (as shown) in which the hemostasis valve 300 may be considered as being open, meaning that there is little to no resistance to advancing an elongate device through the device lumen 308, and a second position in which the hemostasis valve 300 may be considered as being closed, meaning that the hemostasis valve 300 has sealed around the elongate device extending through the device lumen 308. When closed, the hemostasis valve 300 may be considered as sealing against either negative pressure or positive pressure. In some instances, the hemostasis valve 300 may be biased into the first, or open, position. In some instances, the hemostasis valve 300 may be biased into the second, or closed, position. In some instances, the hemostasis valve 300 may not be biased into either position.

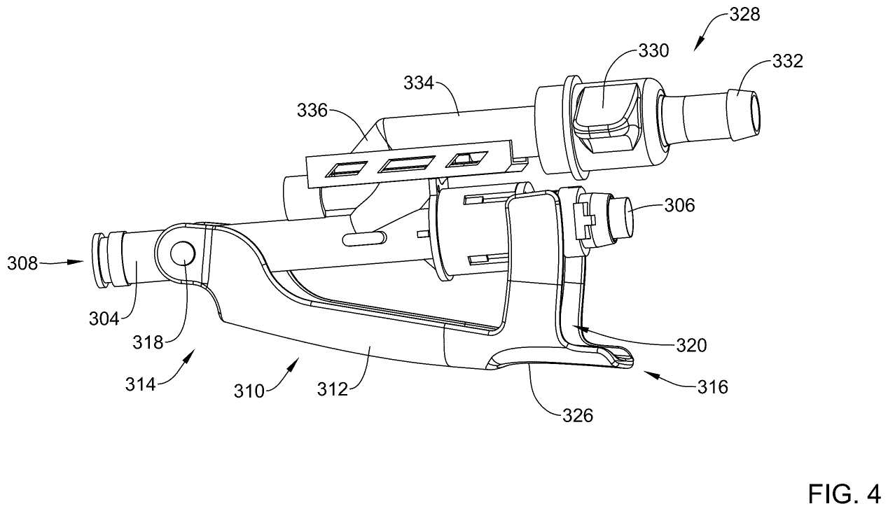

The hemostasis valve 300 may include other components that allow fluids to be introduced into and through the hemostasis valve 300 and into the tubular member 304, for example. In some instances, the hemostasis valve 300 may include a stopcock assembly 328 that may be used to selectively introduce a fluid, or to seal against other fluids. The stopcock assembly 328 may include a stopcock twist 330 and a stopcock glide 332 that may be used to selectively introduce a fluid. While not shown in FIG. 3, the stopcock assembly 328 may include additional structure that provides a selectively open (or closed) fluid coupling between the stopcock glide 332 and the tubular member 304. As seen for example in FIG. 4, in which the housing 302 has been removed, the stopcock assembly 328 may further include a stopcock slide 334 that is fluidly coupled with the stopcock glide 332. In some instances, the stopcock assembly 328 also includes an additional component that is adapted to fluidly couple the stopcock assembly 328 with the tubular member 304.

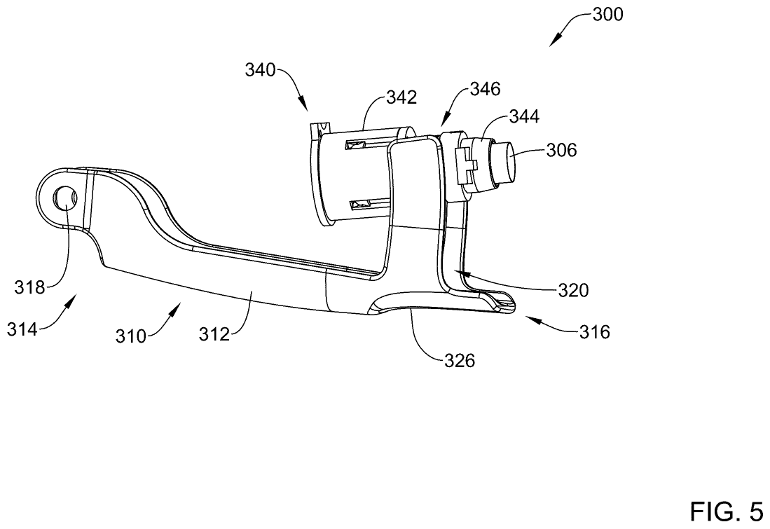

FIG. 5 shows the hemostasis valve 300 with additional components removed to illustrate an actuation mechanism 340. The actuation mechanism 340 may be considered as being the portion of the hemostasis valve 300 that selectively opens and closes the device lumen 308. As will be discussed, the actuation mechanism 340 may be selectively actuated using the lever 310. In some instances, the actuation member 340 includes a first stationary hub 342, a second stationary hub 344 and a rotatable hub 346 that is positioned between the first stationary hub 342 and the second stationary hub 344. Moving the lever 310 may cause the rotatable hub 346 to rotate, thereby actuating the actuation mechanism 340 as will be discussed. The first stationary hub 342, the second stationary hub 344 and the rotatable hub 346 may be formed of any desired material. In some instances, one or more of the first stationary hub 342, the second stationary hub 344 and the rotatable hub 346 may be made from a metal. In some instances, or more of the first stationary hub 342, the second stationary hub 344 and the rotatable hub 346 may be made from a polymeric material. Examples include nylon and ABS (acrylonitrile butadiene styrene). In some instances, lubricious materials may be used. Examples of suitable lubricious materials include POM (polyoxymethylene), also known as acetal, and fluoropolymers such as but not limited to PTFE (polytetrafluoroethylenc).

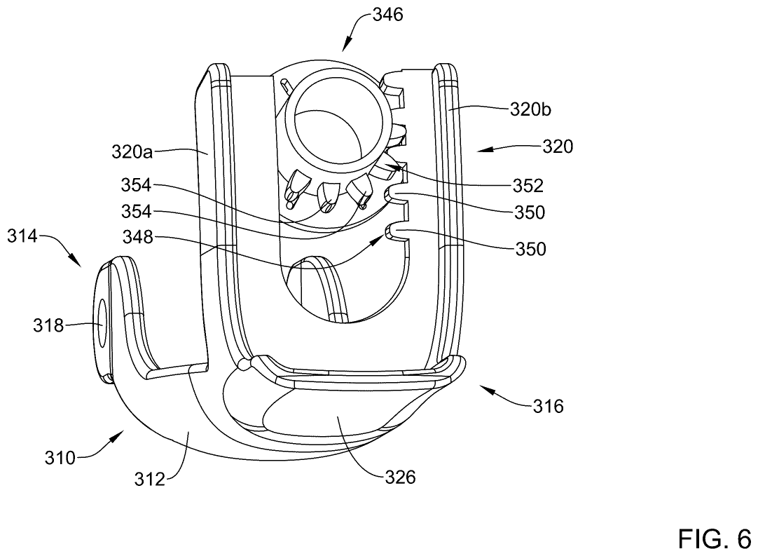

FIG. 6 provides details as to how the lever 310 causes the rotatable hub 346 to rotate. As can be seen, the actuation member 320 of the lever 310 includes a first element 320a and a second element 320b. As shown, the first element 320a does not include a geared surface while the second element 320b includes a geared surface 348 including a number of teeth 350. The rotatable hub 346 includes a hub geared surface 352 including a number of teeth 354. As shown, the teeth 350 forming the geared surface 348 mesh with the teeth 354 forming the hub geared surface 352. As the geared surface 348 moves upward (in the illustrated orientation), the teeth 350 within the geared surface 348 will engage the teeth 354 forming the hub geared surface 352, and will thus cause the rotatable hub 346 to rotate in a counter-clockwise direction.

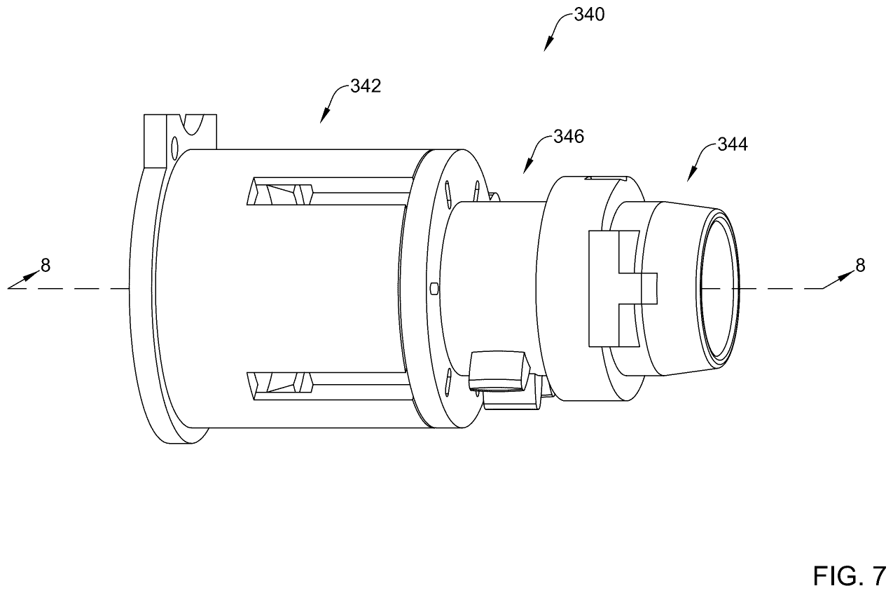

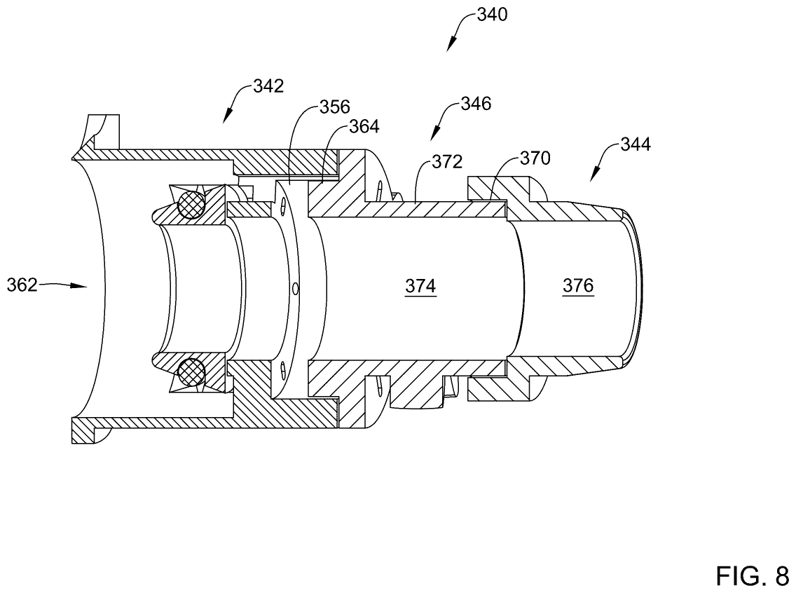

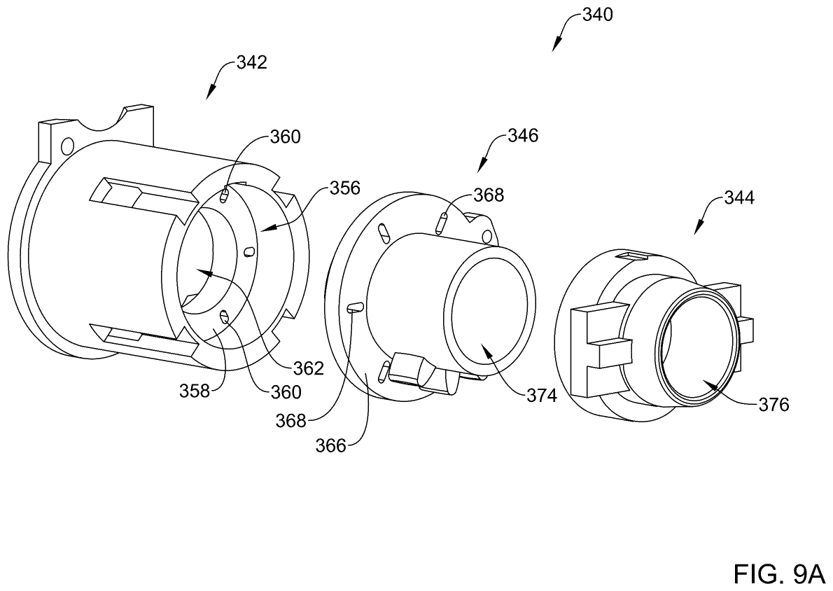

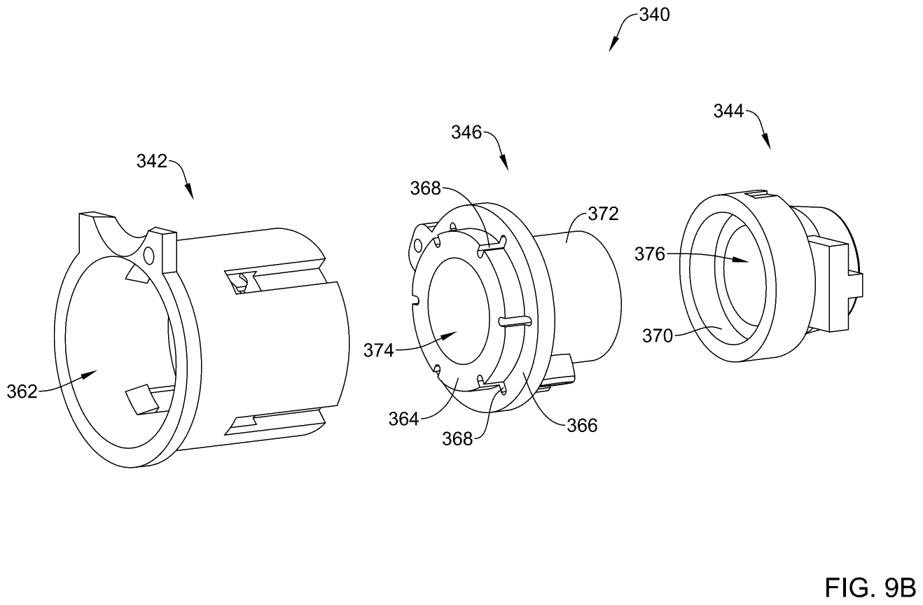

FIG. 7 is a perspective view of the actuation mechanism 340 and FIG. 8 is a cross-sectional view thereof taken along the line 8-8 of FIG. 7. FIG. 9A is a first exploded perspective view of the actuation mechanism 340 and FIG. 9B is a second exploded perspective view of the actuation mechanism 340. As noted, the actuation member 340 includes the first stationary hub 342, the second stationary hub 344 and the rotatable hub 346 that is disposed between the first stationary hub 342 and the second stationary hub 344. In some instances, the first stationary hub 342 and the second stationary hub 344 support the rotatable hub 346 in such a way as to allow the rotatable hub 346 to rotate when driven into rotation via the lever 310.

The first stationary hub 342 includes an annular recess 356 that includes a bottom surface 358. In some instances, the bottom surface 358 may be considered as being a face of the first stationary hub 342. The bottom surface 358 includes a number of apertures 360 that may be used to accommodate a number of cord segments, as will be discussed. The first stationary hub 342 includes a first stationary hub lumen 362. The rotatable hub 346 includes an annular protrusion 364 that is adapted to fit within the annular recess 356 of the first stationary hub 342. In some instances, the annular recess 346 of the first stationary hub 342 may be considered as supporting the rotatable hub 346 in a way that allows the annular protrusion 364 of the rotatable hub 346 to rotate relative to the annular recess 356 of the first stationary hub 342. The rotatable hub 346 includes an annular periphery 366. The annular periphery 366 includes a number of apertures 368 that may be used to accommodate a number of cord segments, as will be discussed. The annular periphery 366 may be considered as defining a face of the rotatable hub 346.

The second stationary hub 344 includes an annular recess 370 that is adapted to accommodate a cylindrical body portion 372 of the rotatable hub 346. In some instances, the annular recess 370 of the second stationary hub 344 may be considered as supporting the rotatable hub 346 in a way that allows the cylindrical body portion 372 of the rotatable hub 346 to rotate relative to the annular recess 370 of the second stationary hub 344. Accordingly, together the first stationary hub 342 and the second stationary hub 344 support the rotatable hub 346 and allow the rotatable hub 346 to rotate when actuated via the lever 310. The rotatable hub 346 includes a rotatable hub lumen 374 and the second stationary hub 344 includes a second stationary hub lumen 376. The first stationary hub lumen 362, the rotatable hub lumen 374 and the second stationary hub 344 are axially aligned such that the polymeric member 306 (shown in FIG. 3) is able to extend through the actuation mechanism 340 and provide part of the device lumen 308.

As noted, a number of cord segments may extend between a face of the first stationary hub 342 (such as the bottom surface 358 of the annular recess 356) and a face of the rotatable hub 346 (such as the annular periphery 366). The face of the first stationary hub 342 and the face of the rotatable hub 346 may face each other, such that the apertures 360 within the bottom surface 358 and the apertures 368 within the annular periphery 366 face each other. A number of cord segments may be run between the face of the first stationary hub 342 and the opposing face of the rotatable hub 346. The cord segments may be considered as extending external of the polymeric member 306.

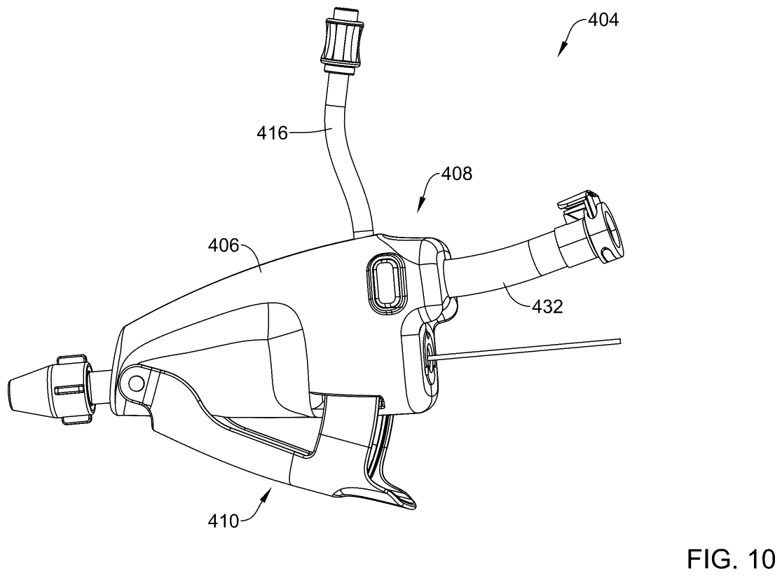

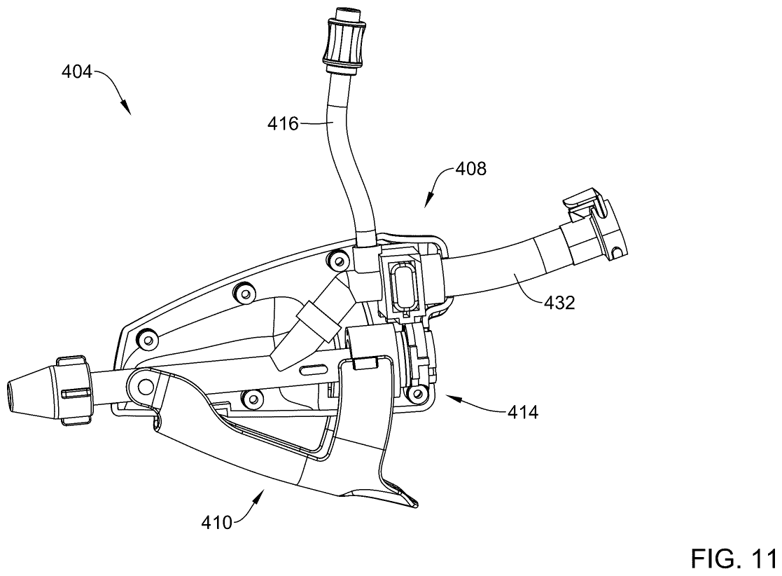

FIG. 10 is a perspective view of an illustrative hemostasis valve 404 and FIG. 11 shows the illustrative hemostasis valve 404 with a portion of a housing 406 removed. The hemostasis valve 404 includes a lever 410 that is adapted to engage an actuation mechanism 414 within the housing 406 in order to selectively close the hemostasis valve 404. The hemostasis valve 404 has additional fluid handling structure 408 that can accommodate a fluid inlet 416 and a fluid inlet 432. There are also differences with respect to the actuation mechanism 414.

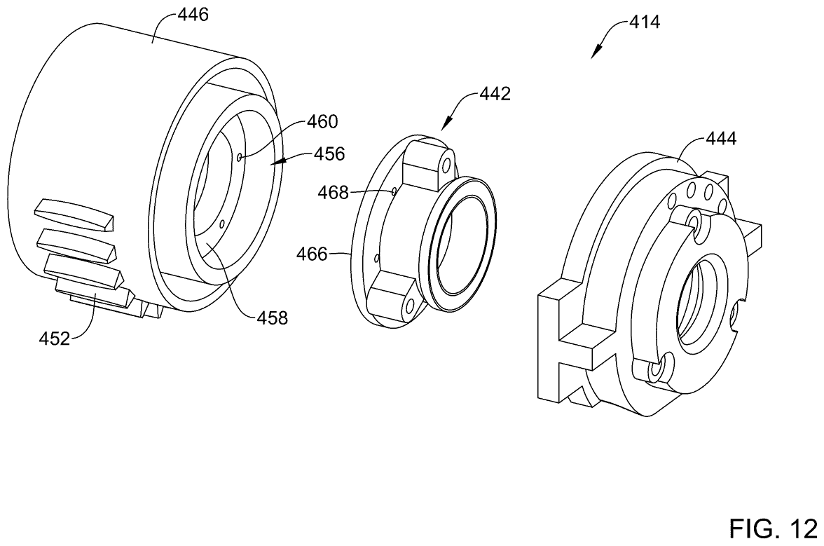

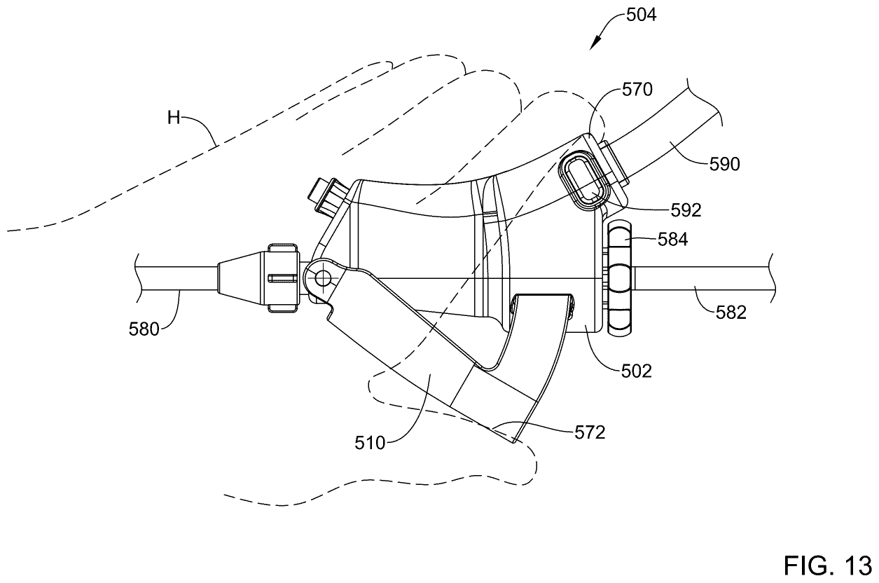

FIG. 12 is an exploded perspective view of the actuation mechanism 414, showing a rotatable hub 446 and a stationary hub 442. A hub 444 serves to locate the stationary hub 442. The rotatable hub 446 includes a geared surface 452 that is adapted to be engaged by the lever 410. The rotatable hub 446 includes an annular recess 456 having a bottom surface 458 bearing a number of apertures 460 that are adapted to accommodate a number of cord segments. The stationary hub 442 includes a surface 466 bearing a number of apertures 468 to accommodate a number of cord segments. In some instances, the surface 466 is adapted to fit within the annular recess 456 of the rotatable hub 446.

FIG. 13 is a schematic view of an illustrative hemostasis valve 500. The illustrative hemostasis valve 500 includes a housing 502 and a lever 510 that are being held within a hand H. An index finger of the hand H fits within a recessed region 570 while a thumb of the hand H fits within a recessed region 572 of the lever 510. The hemostasis valve 500 is attached to an elongate shaft 580 that forms a device lumen. An elongate device 582 is extending through the hemostasis valve 500. In some instances, as shown, the hemostasis valve 500 may include a mechanical feature 584 that may be rotated in one direction to loosen access for the elongate device 582 and may be rotated in an opposing direction to restrict access for the elongate device 582. In some instances, the mechanical feature 584 functions as a third hand that can be used to lock the elongate device 582 in position. The hemostasis valve 500 may include a fluid line 590 that may be used to provide fluid. A button 592 may open or close fluid flow from the fluid line 590. While particular housing designs, including particular fluid handling capabilities have been shown, it will be appreciated that modifications may be incorporated.

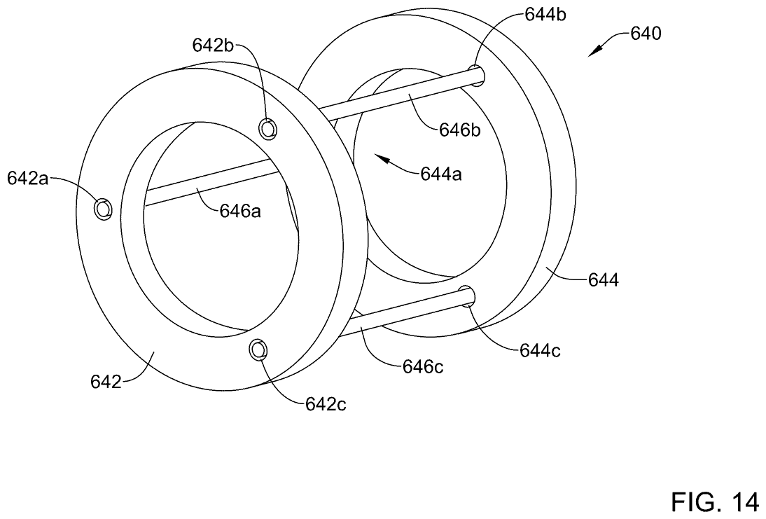

FIG. 14 is a perspective view of an illustrative actuation mechanism 640. The illustrative actuation mechanism 640 may be considered as being an example of the actuation mechanism 340 shown in FIGS. 5, 7 and 8, for example. The actuation mechanism 640 includes a first annular member 642 and a second annular member 644. The first annular member 642 may be considered as representing the bottom surface 358 within the first stationary hub 342 and the second annular member 644 may be considered as representing the annular periphery 366 of the rotatable hub 346, for example. The actuation mechanism 640 includes several cord segments 646a, 646b and 646c each extending from corresponding apertures 642a, 642b and 642c formed within the first annular member 642 and apertures 644a, 644b and 644c formed within the second annular member 644, although the aperture 644a is not visible in this view. A total of three cord segments 646a, 646b and 646c are shown, although in some instances the actuation mechanism 640 may include a greater number of cord segments. As an example, the actuation mechanism 640 may include a total of six cord segments. The cord segments 646a, 646b and 646c may be circumferentially spaced about and in some cases may be equidistantly spaced apart. As an example, the three cord segments 646a, 646b and 646c may be spaced about 120 degrees apart. In an example with a total of six cord segments, each cord segment may be spaced about 60 degrees apart.

In some instances, each cord segment 646a, 646b and 646c may be part of a single cord that is wound back and forth between the first annular member 642 and the second annular member 644. As an example, a single cord may be wrapped back and forth between the first annular member 642 and the second annular member 644 to form all of the cord segments 646a, 646b and 646c, for example, and then the two ends of the single cord may be fastened in place. In some instances, the two ends may be terminated together by tying and gluing the two ends together. In some instances, using a single cord to form each of the cord segments 646a, 646b and 646c may provide manufacturing benefits including tolerance.

In some instances, each cord segment 646a, 646b and 646c may be a separate cord that extends from the first annular member 642 and the second annular member 644. The cord segments 646a, 646b and 646c may be made of a variety of different materials, particularly if those materials are low stretch, pliable and strong. In some instances, each cord segment 646a, 646b and 646c may be a different material. In some instances, each cord segment 646a, 646b and 646c may be made of the same material.

In some instances, the cord segments 646a, 646b and 646c may be polymer coated stainless steel cables. In some instances, the cord segments 646a, 646b and 646c may be made have a nitrile or urethane sleeve over them. In some instances, the cord segments 646a, 646b and 646c may be made of polyethylene microfiber materials including those used in making high tech fishing line. The cord segments 646a, 646b and 646c may be a monofilament line, and/or may be woven. In some instances, suture material may be used in forming the cord segments 646a, 646b and 646c.

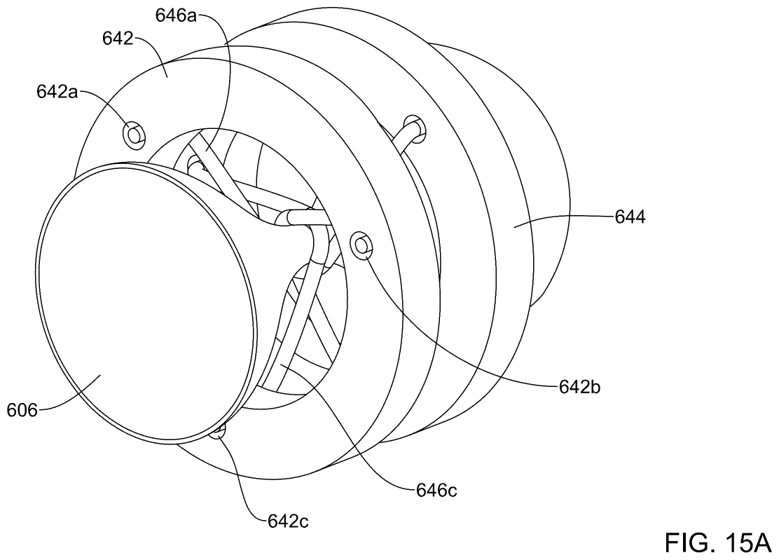

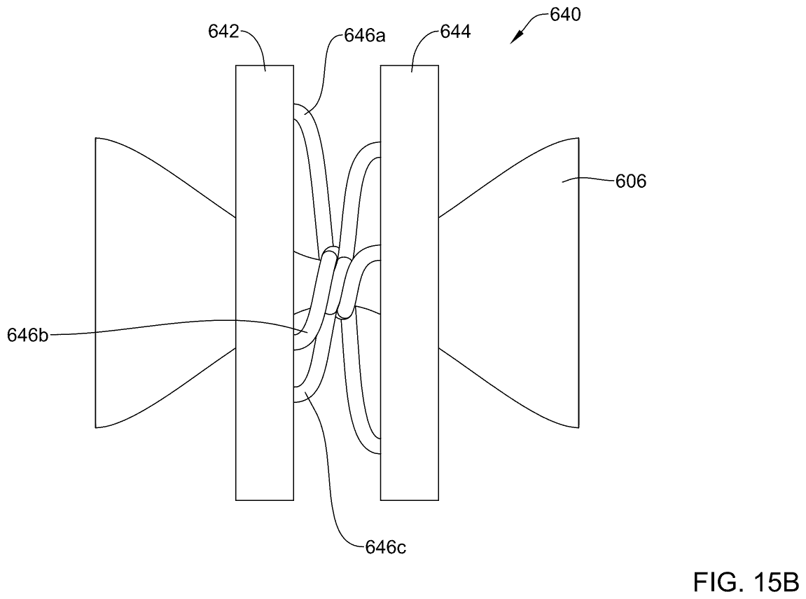

As shown in FIG. 14, the actuation mechanism 640 is shown in an initial configuration prior to assembly of a hemostasis valve including the actuation mechanism 640. As seen, the cord segments 646a, 646b and 646c are each parallel or substantially parallel with each other, where substantially parallel is defined as being within ten degrees of being parallel. When the actuation mechanism 640 is installed into a hemostasis valve such as the hemostasis valve 300, one or both of the first annular member 642 and the second annular member 644 are rotated relative to each other, which causes the cord segments 646a, 646b and 646c to twist. FIG. 15A is a perspective view of the actuation mechanism 640 as it would be oriented within a hemostasis valve, with the cord segments 646a, 646b and 646c wrapped around a polymeric member 606 and FIG. 15B is a corresponding side view.

As can be seen, relative rotation between the first annular member 642 and the second annular member 644 has resulted in the cord segments 646a, 646b and 646c twisting down towards a centerline, thereby constricting the polymeric member 606. The polymeric member 606 has a maximum diameter (its original, unbiased diameter) at either end of the polymeric member 606 and a minimum diameter where the cord segments 646a, 646b and 646c engage the polymeric member 606. In some instances, the illustrated constriction of the polymeric member 606 may represent a degree of constriction that corresponds to having assembled the hemostasis valve, and the polymeric member 606 may be further constricted by further relative rotation between the first annular member 642 and the second annular member 644. In some instances, the illustrated constriction of the polymeric member 606 may represent a degree of constriction resulting from further relative rotation between the first annular member 642 and the second annular member 644 to seal against an elongate device extending through the polymeric member 606.

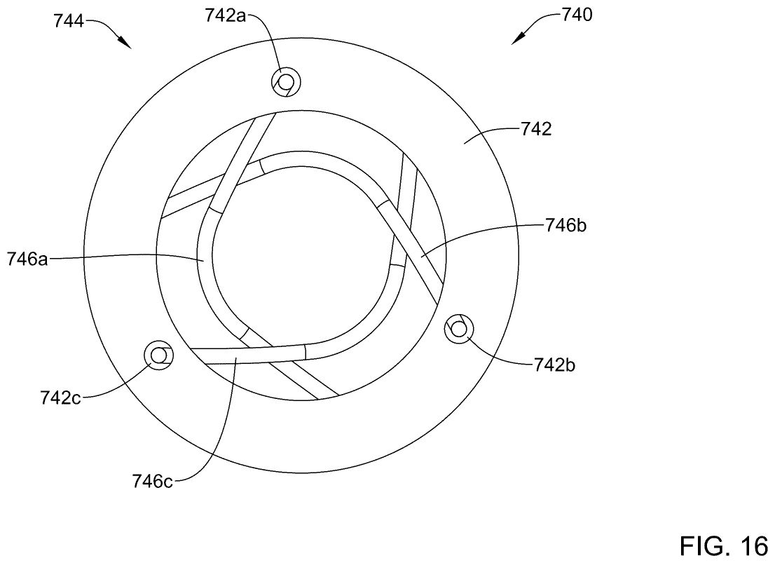

FIGS. 15A and 15B show the actuation mechanism 640 having a small opening formed between the cord segments 646a, 646b and 646c. FIG. 16 is an end view of an illustrative actuation member 740 that may be considered as having a large opening. The illustrative actuation mechanism 740 may be considered as being an example of the actuation mechanism 340 shown in FIGS. 5, 7 and 8, for example. The actuation mechanism 740 includes a first annular member 742 and a second annular member 744 (behind the first annular member 742 in the illustrated orientation). The corresponding polymeric member is not shown. The first annular member 742 may be considered as representing the bottom surface 358 within the first stationary hub 342 and the second annular member 744 may be considered as representing the annular periphery 366 of the rotatable hub 346, for example. The actuation mechanism 740 includes several cord segments 746a, 746b and 746c each extending from corresponding apertures 742a, 742b and 742c formed within the first annular member 742 and corresponding apertures (not shown) formed within the second annular member 744. A total of three cord segments 746a, 746b and 746c are shown, although in some instances the actuation mechanism 740 may include a greater number of cord segments. As an example, the actuation mechanism 740 may include a total of six cord segments.

In some instances, each cord segment 746a, 746b and 746c may be part of a single cord that is wound back and forth between the first annular member 742 and the second annular member 744. In some instances, each cord segment 746a, 746b and 746c may be a separate cord that extends from the first annular member 742 and the second annular member 744. The cord segments 746a, 746b and 746c may be made of a variety of different materials, particularly if those materials are low stretch, pliable and strong. In some instances, the cord segments 746a, 746b and 746c may be polymer coated stainless steel cables. In some instances, the cord segments 746a, 746b and 746c may be made have a nitrile or urethane sleeve over them. In some instances, the cord segments 746a, 746b and 746c may be made of polyethylene microfiber materials including those used in making high tech fishing line. The cord segments 746a, 746b and 746c may be a monofilament line, and/or may be woven. In some instances, suture material may be used in forming the cord segments 746a, 746b and 746c.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A hemostasis valve adapted to allow an elongate device to pass through the hemostasis valve while sealing against the elongate device, the hemostasis valve comprising:
a housing, the housing surrounding:
a first hub having a first face;
a second hub having a second face, the second hub arranged such that the second face faces the first face;
an elastomeric tube extending between the first hub and the second hub, the elastomeric tube including an outer surface and an inner surface defining part of a device lumen extending through the housing; and
a plurality of cord segments extending from the first face to the second face, each of the plurality of cord segments circumferentially spaced from neighboring cord segments; and
wherein relative rotation between the first hub and the second hub causes each of the plurality of cord segments to cinch down onto the outer surface of the elastomeric tube and compress the elastomeric tube down into the device lumen, thereby sealing between the inner surface of the elastomeric tube and any elongate device extending through the device lumen.

2. The hemostasis valve of claim 1, wherein the first hub is stationary.

3. The hemostasis valve of claim 1, wherein the first hub comprises an annular recess having a bottom surface, the bottom surface defining the first face.

4. The hemostasis valve of claim 3, wherein the second hub is adapted to be rotated.

5. The hemostasis valve of claim 4, wherein the second hub comprises:
an annular protrusion that is adapted to extend into the annular recess of the first hub; and
an annular periphery adjacent the annular protrusion, the annular periphery defining the second face.

6. The hemostasis valve of claim 4, wherein the second hub comprises a hub geared surface.

7. The hemostasis valve of claim 6, further comprising a lever including an elongate lever body extending from a first region to a second region;
the first region pivotably secured to the housing;
the second region including an actuation member extending away from the elongate lever body and extending into the housing;
the actuation member including an actuation member gear surface adapted to engage the hub geared surface such that moving the lever relative to the housing causes rotation of the second hub as the actuation member gear surface translates relative to the hub gear surface.

8. The hemostasis valve of claim 7, wherein the lever is movable between a first position in which the second hub remains stationary and a second position in which the second hub is caused to rotate.

9. The hemostasis valve of claim 8, wherein the lever is biased to the first position.

10. The hemostasis valve of claim 8, wherein the hemostasis valve exhibits minimal insertion force for inserting the elongate device into the lumen when the lever is in the first position.

11. The hemostasis valve of claim 5, further comprising a third hub positioned such that the second hub is disposed between the first hub and the third hub, the second hub further comprising a cylindrical body that extends into an annular void formed within the third hub;
wherein the second hub rotates between the first hub and the third hub.

12. The hemostasis valve of claim 1, wherein each of the plurality of cord segments comprise a portion of one or more cords extending back and forth between the first hub and the second hub.

13. A hemostasis valve, comprising:
a stationary hub including:
an annular recess having a bottom surface; and
a stationary hub lumen extending through the stationary hub;
a rotatable hub including:
an annular protrusion that extends into the annular recess;
an annular periphery adjacent the annular protrusion;
a rotatable hub lumen extending through the rotatable hub lumen;
a plurality of cord segments extending between the bottom surface of the stationary hub and the annular periphery of the rotatable hub; and
a polymeric member extending through the stationary hub lumen and the rotatable hub lumen, the polymeric member defining a device lumen extending therethrough;
wherein rotating the rotatable hub causes each of the plurality of cord segments to cinch down onto the polymeric member and compress the polymeric member down into the device lumen, thereby sealing between an inner surface of the polymeric member and any elongate device extending through the lumen.

14. The hemostasis valve of claim 13, wherein the bottom surface of the annular recess comprises a plurality of apertures adapted to accept each of the plurality of cords.

15. The hemostasis valve of claim 13, wherein the annular periphery comprises a plurality of apertures adapted to accept each of the plurality of cords.

16. The hemostasis valve of claim 13, further comprising a housing that houses the stationary hub, the rotatable hub, the plurality of cords and the polymeric member.

17. The hemostasis valve of claim 16, wherein the rotatable hub further comprises a hub geared surface.

18. The hemostasis valve of claim 17, further comprising:
a lever including an elongate lever body extending from a first end to a second end;
the first end pivotably secured to the housing;
the second end including an actuation member extending away from the elongate lever body and extending into the housing;
the actuation member including an actuation member gear surface adapted to engage the hub geared surface such that moving the lever relative to the housing causes rotation of the second hub as the actuation member gear surface translates relative to the hub gear surface.

19. A hemostasis valve, comprising:
a first stationary hub;
a second stationary hub spaced from the first stationary hub;
a rotatable hub rotatably supported by the first stationary hub and the second stationary hub;
one or more cords extending back and forth between the first stationary hub and the rotatable hub; and
a polymeric member extending through the first stationary hub, the second stationary hub and the rotatable hub, the polymeric member defining a device lumen extending therethrough;
wherein rotating the rotatable hub causes each of the plurality of cords to cinch down onto the polymeric member and compress the polymeric member down into the device lumen, thereby sealing between an inner surface of the polymeric member and any elongate device extending through the lumen.

20. The hemostasis valve of claim 19, further comprising a lever that, when actuated, causes the rotatable hub to rotate.
